(19) United States
(12) Patent Application Publication
(10) Pub. No.: US 2010/0318175 A1
(43) Pub. Date: Dec. 16, 2010

(54) VASCULAR GRAFT PROSTHESIS WITH SELECTIVE FLOW REDUCTION

(75) Inventors: Enrique Abarca, Chandler, AZ (US); Andrzej J. Chanduszko, Chandler, AZ (US)

Correspondence Address: C. R. Bard, Inc.;Bard Peripheral Vascular, Inc., 1415 W. 3rd Street, P.O. Box 1740, Tempe, AZ, 85280-1740, US

(73) Assignee: C.R. Bard, Inc., Murray Hill, NJ (US)

(21) Appl. No.: 12/811,291

(22) PCT Filed: Dec. 30, 2008
(86) PCT No.: PCT/US08/88556

§ 371 Date: Aug. 20, 2010

Related U.S. Application Data
(60) Provisional application No. 61/009,755, filed on Dec. 31, 2007.

(51) Int. Cl. A61F 2/82

(52) U.S. Cl. 623/1.13; 623/1.18

(57) ABSTRACT

A vascular graft prosthesis comprising a tubular member having a luminal wall defining a lumen and an inner luminal diameter; and means for non-invasively constricting at least a portion of the luminal wall to reduce the inner luminal diameter and reduce a volumetric flow rate through the tubular member at the site of implantation of the vascular graft prosthesis, and during normal operating conditions, the means for constricting being operable to therefore facilitate an increase in a volumetric flow rate through the target limb to treat various symptoms manifesting themselves as a result of the implanted graft prosthesis, such as those resulting from an arteriovenous graft access and indicative of Steal syndrome.

### PRIORITY

This application claims the benefit of U.S. Provisional Patent Application No. 61/009,755, filed Dec. 31, 2007, which is incorporated by reference in its entirety into this application.

### FIELD

The present invention relates generally to vascular grafts or vascular graft prostheses, and more particularly to vascular graft prostheses such as those intended for use to alleviate or treat peripheral vascular disease (e.g., peripheral bypass grafts', as well as those intended for hemodialysis access (e.g., arteriovenous (A/V) access grafts).

### BACKGROUND

Vascular graft prostheses represent a very common class of biocompatible prosthetic implants used for a variety of purposes. For example, peripheral bypass grafts represent a specific type of vascular graft intended to treat peripheral artery occlusive disease (PAOD) (also known as peripheral vascular disease (PVD) and peripheral artery disease (PAD)), which describes the condition where the large peripheral arteries are stenosed or occluded. Peripheral bypass grafting is generally understood to describe the procedure in which an artificial vascular graft prosthesis is used to circumvent a stenosed or occluded area of the arterial vasculature. In another example, hemodialysis access grafts, or arteriovenous access grafts, comprise another specific type of vascular graft intended to provide hemodialysis “access” for patients suffering from renal disease, such as renal artery stenosis, or renal dysfunction or failure.

Renal disease is a life threatening condition in which the kidneys' ability to function properly is considerably diminished, or in some cases, in which the kidneys fail to function altogether. Although affecting millions of people worldwide, renal disease is treatable. The most common treatment for renal disease is hemodialysis, which comprises a complicated and imposing method for filtering blood to remove waste products, such as potassium and urea, as well as free water. Critical removal of these waste products is achieved in a similar manner as other dialysis techniques, namely by diffusion of solutes across a semi-permeable membrane.

Hemodialysis treatment involves the filtering of blood through a dialyzer, and therefore, requires access to the circulatory system. Hemodialysis for patients typically comprises the utilization of one or more modes of access, namely catheter access (a synthetic device used to allow large flows of blood to be withdrawn from one lumen, to go into the dialysis circuit, and to be returned via the other lumen), arteriovenous fistula access (direct anastomosis of an artery and a vein, at least partially bypassing capillaries), and arteriovenous graft access (anastomosis of an artery and a vein using a prosthetic graft, sometimes referred to as an access graft, also at least partially bypassing capillaries). With specific focus herein, an A/V access graft prosthesis for use in providing arteriovenous access may be more specifically described as a subcutaneous prosthetic device used to establish a fluid communication with, and to effectively provide an access to the patient's circulatory system. Arteriovenous access provides a connection or region of high blood flow, and needles operable with a dialysis machine may be inserted into the A/V graft prosthesis to access this region and to facilitate hemodialysis treatment. Arteriovenous graft access typically represents the alternative choice to arteriovenous fistula access for facilitating hemodialysis, with arteriovenous graft access most often being used when the patient's native vasculature does not permit direct artery to vein anastomosis and formation of a fistula. However, arteriovenous graft access provides some advantages over arteriovenous fistula access. For example access graft prostheses have been found to mature faster than fistulas, often being ready for use several weeks after formation.

Arteriovenous graft access represents a long-term access solution, with the A/V access graft prosthesis being left implanted subcutaneously for as long as the graft remains patent, and as long as thrombosis is averted. Use of an arteriovenous access graft to form an arteriovenous access intentionally functions to divert blood from an artery to a vein to provide access to a region of high blood flow for hemodialysis treatment. However, understandably so, this also effectively functions to reduce blood flow through the artery, resulting in an insufficient blood supply to the lower sections of the subject limb (the limb having and supporting the arteriovenous access formed therein). Indeed, one significant drawback to prior related access graft prostheses is that no provision is made to control blood flow through the graft, and therefore to control blood flow through the artery anastomosed to the graft.

One particular risk associated with arteriovenous access sites is the potential for the onset of vascular access steal syndrome, which describes a condition of vascular insufficiency as a result of a formed arteriovenous access. In the event blood flow rates through the arteriovenous access are too high, and the vasculature that supplies the rest of the subject limb is insufficient, inordinate amounts of blood entering the subject limb may be drawn through the arteriovenous access via the A/V access graft prosthesis and returned to the general circulation without entering the capillaries of the subject limb. With this condition, various symptoms may be manifested, such as pallor, a diminished pulse, necrosis, a decreased wrist-brachial index, coldness in the extremities of the subject limb, cramping pains, and if the vascular insufficiency is severe enough, possible tissue damage. Existing treatments to alleviate these symptoms and to treat steal syndrome involve access ligation or banding of the graft or a vessel distal to the graft to restrict blood flow through the graft. However, each of these procedures is highly invasive, and involves considerable risk. In addition, ligation and banding are each semi-permanent, in that they are incapable of being relaxed or further constricted without taking additional invasive measures.

### SUMMARY OF THE INVENTION

In light of the problems and deficiencies inherent in the prior art, the present invention seeks to overcome these by providing noninvasive, selective control (e.g., selective reduction and/or increase) of blood flow (e.g., volumetric blood flow) through an arteriovenous or vascular access graft prosthesis used to form an arteriovenous access site, thus selectively facilitating an increase in blood flow through a subject artery anastomosed to the A/V access graft prosthesis used to form the arteriovenous access site.

In accordance with the invention as embodied and broadly described herein, the present invention resides in a vascular graft prosthesis implanted within a subject limb and anastomosed to a target vessel to facilitate at least partial diversion of flow from the target, the vascular graft prosthesis comprising: (a) a tubular member having a luminal wall defining a lumen and an inner luminal diameter; and (b) means for constricting at least a portion of the luminal wall to reduce the inner luminal diameter and reduce a volumetric flow rate through the tubular member at the site of implantation, and during normal operating conditions, the means for constricting being operable to therefore facilitate an increase in volumetric flow rate through the target vessel.

The present invention also resides in an arteriovenous access graft prosthesis for use in forming an artificial arteriovenous graft access for facilitating hemodialysis treatment, the arteriovenous access graft prosthesis comprising: (a) a tubular member having a luminal wall defining a lumen and an inner luminal diameter; (b) an arterial end operable with the tubular member to facilitate arterial anastomosis to a target artery; (c) a venous end operable with the tubular member to facilitate venous anastomosis to a target vein; and (d) means for constricting at least a portion of the luminal wall to reduce the inner luminal diameter and reduce a volumetric flow rate through the tubular member at the site of implantation, and during normal operating conditions, the means for constricting being operable to therefore facilitate an increase in a volumetric flow rate through the target artery.

The present invention further resides in a method for regulating blood flow through an arteriovenous access graft prosthesis, and ultimately through a target artery, forming an arteriovenous graft access for hemodialysis treatment, the method comprising: (a) locating the arteriovenous graft access and the arteriovenous access graft prosthesis anastomosed to a target artery and a target vein, the arteriovenous access graft prosthesis comprising a tubular member having a luminal wall defining a lumen and an inner luminal diameter, and means for constricting at least a portion of the luminal wall to reduce the inner luminal diameter and reduce a volumetric flow rate through the tubular member at the site of implantation, and during normal operating conditions; and (b) exposing the means for constricting to a stimulus sufficient to cause at least partial transitioning of the means for constricting from a radially expanded diameter configuration to a radially reduced diameter configuration, thereby reducing the inner luminal diameter and decreasing a volumetric flow rate through the arteriovenous access graft prosthesis, and increasing a volumetric flow rate through the target artery.

The present invention still further resides in a method for facilitating regulation of blood flow through a vascular graft prosthesis, and a target artery, the method comprising: (a) forming a tubular member having a luminal wall defining a lumen and an inner luminal diameter; and (b) relating the tubular member with means for constricting at least a portion of the luminal wall to reduce the inner luminal diameter and reduce a volumetric flow rate through the tubular member at the site of implantation, and during normal operating conditions, the means for constricting being adapted to transition from a radially expanded diameter configuration to a radially reduced diameter configuration, thereby reducing the inner luminal diameter and decreasing a volumetric flow rate through the vascular graft prosthesis, and increasing a volumetric flow rate through the target artery.

The present invention still further resides in a vascular implantation device comprising a constricting element comprising, at least in part, a thermoresponsive smart material makeup; and a plurality of carbon nanotubes operable with the constricting element to generate thermal energy upon being exposed to an external stimulus, the thermal energy activating the smart material and causing the constricting element to at least partially transition from a deformed configuration to a remembered configuration.

### BRIEF DESCRIPTION OF THE DRAWINGS

The present invention will become more fully apparent from the following description and appended claims, taken in conjunction with the accompanying drawings. Understanding that these drawings merely depict exemplary embodiments of the present invention they are, therefore, not to be considered limiting of its scope. It will be readily appreciated that the components of the present invention, as generally described and illustrated in the figures herein, could be arranged and designed in a wide variety of different configurations. Nonetheless, the invention will be described and explained with additional specificity and detail through the use of the accompanying drawings in which:

FIG. 1 illustrates a graphical representation of a human arm, showing a portion of the vasculature having an artificial arteriovenous access formed using an arteriovenous access graft prosthesis configured in accordance with one exemplary embodiment of the present invention, wherein the arteriovenous access graft prosthesis comprises a constricting element, having a smart material makeup, operable with the elongate tubular member to constrict a portion thereof for the purpose of reducing the inner luminal diameter and the volumetric flow of fluids (e.g., blood) through the tubular member;

FIG. 2 illustrates a partial, detailed perspective view of an arteriovenous access graft prosthesis formed in accordance with one exemplary embodiment of the present invention, wherein the arteriovenous access graft prosthesis comprises a constricting element, having a smart material makeup, disposed circumferentially about an exterior surface of the A/V access graft prosthesis, the constricting element having the form of an annular band extending only partially along the longitudinal length of the tubular member;

FIG. 3 illustrates a partial, detailed cross-sectional side view of the arteriovenous access graft prosthesis of FIG. 2 taken along lines 3-3;

FIG. 4 illustrates a partial, detailed side view of the arteriovenous access graft prosthesis of FIG. 2, with the constricting element being shown in a remembered configuration (a shrunk configuration) as a result of an external stimulus being applied thereto, which external stimulus causes the constricting element to activate and transition or shift from a deformed, radially expanded diameter state or configuration to a remembered, radially reduced diameter state or configuration, thus constricting the tubular member and reducing its interior luminal diameter;

FIG. 5 illustrates a partial, detailed cross-sectional end view of the arteriovenous access graft prosthesis of FIG. 2, taken along lines 5-5 of FIG. 4;

FIG. 6 illustrates a partial, detailed perspective view of an arteriovenous access graft prosthesis formed in accordance with one exemplary embodiment of the present invention, wherein the arteriovenous access graft prosthesis comprises a constricting element, having a smart material makeup, disposed circumferentially between layers of the tubular member of the graft prosthesis, the constricting element having the form of an annular band extending only partially along the longitudinal length of the tubular member;

FIG. 7 illustrates a partial, detailed cross-sectional side view of the arteriovenous access graft prosthesis of FIG. 6 taken along lines 7-7;

FIG. 8 illustrates a partial, detailed side view of the arteriovenous access graft prosthesis of FIG. 6, with the constricting element being shown in a remembered configuration (a shrunk configuration) as a result of an external stimulus being applied thereto, which external stimulus causes the constricting element to activate and transition or shift from a deformed state or configuration to the remembered state or configuration, thus constricting the tubular member and reducing its interior luminal diameter;

FIG. 9 illustrates a partial, detailed cross-sectional end view of the arteriovenous access graft prosthesis of FIG. 6, taken along lines 9-9 of FIG. 8;

FIG. 10 illustrates a partial, detailed perspective view of an arteriovenous access graft prosthesis formed in accordance with another exemplary embodiment of the present invention, wherein the arteriovenous access graft prosthesis comprises a plurality of constricting elements operable to selectively reduce the inner luminal diameter of the tubular member, and to provide a plurality of selectable zones or reduced diameter states;

FIG. 11 illustrates a partial cross-sectional side view of the arteriovenous access graft prosthesis of FIG. 10, taken along lines 11-11;

FIG. 12 illustrates a partial cross-sectional side view of the arteriovenous access graft prosthesis of FIG. 10, wherein a first constricting element is shown in a radially reduced diameter configuration;

FIG. 13 illustrates a partial cross-sectional side view of the arteriovenous access graft prosthesis of FIG. 12, wherein a second constricting element is shown in a radially reduced diameter configuration;

FIG. 14 illustrates a partial, detailed cross-sectional side view of an arteriovenous access graft prosthesis formed in accordance with another exemplary embodiment of the present invention, wherein the arteriovenous access graft prosthesis comprises a constricting element operable to selectively reduce the inner luminal diameter of the tubular member, and wherein the luminal wall of the tubular member comprises a plurality of apertures configured to minimize anomalies within the luminal wall and along the inner luminal surface caused by the transformation of the constricting element to a radially reduced diameter state;

FIG. 15 illustrates a partial, detailed cross-sectional side view of the arteriovenous access graft prosthesis of FIG. 14, with the apertures shown in a collapsed state;

FIG. 16 illustrates a partial, detailed side view of an arteriovenous access graft prosthesis formed in accordance with another exemplary embodiment of the present invention, wherein the arteriovenous access graft prosthesis comprises a plurality of wire-like constricting elements oriented transverse to the longitudinal axis of the tubular member, the plurality of constricting elements operating in concert to constrict the inner luminal diameter of the tubular member;

FIG. 17 illustrates a partial, detailed cross-sectional side view of an arteriovenous access graft prosthesis formed in accordance with another exemplary embodiment of the present invention, wherein the arteriovenous access graft prosthesis comprises a plurality of carbon nanotubes embedded within the smart material composition making up the constricting element; and

FIG. 18 illustrates a partial, detailed cross-sectional view of an arteriovenous access graft prosthesis formed in accordance with another exemplary embodiment of the present invention, wherein the constricting element is disposed within the lumen of the tubular member along the inner luminal surface.

### DETAILED DESCRIPTION OF EXEMPLARY EMBODIMENTS

The following detailed description of exemplary embodiments of the invention makes reference to the accompanying drawings, which form a part hereof and in which are shown, by way of illustration, exemplary embodiments in which the invention may be practiced. While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only to describe the features and characteristics of the present invention, to set forth the best mode of operation of the invention, and to sufficiently enable one skilled in the art to practice the invention. Accordingly, the scope of the present invention is to be defined solely by the appended claims.

The following detailed description and exemplary embodiments of the invention will be best understood by reference to the accompanying drawings, wherein the elements and features of the invention are designated by numerals throughout.

The term “smart material” is representative of various different material types commonly known in the art, and is intended herein to be interpreted as generally understood. For purposes of discussion, smart or intelligent materials comprise shape memory or shape shifting materials that have one or more properties that can be significantly altered or manipulated using external stimuli, such as stress, temperature, moisture, pH, electric or magnetic fields, radiation, etc. to cause or induce deformation. Smart materials include, but are not necessarily limited to, shape memory alloys (SMAs), shape memory polymers (SMPs) (including shape-shifting polymer gels), piezoelectric materials, and magnetic shape memory alloys. It is further contemplated that the smart materials may comprise one-way (having a single remembered configuration, with deformation being required to create a non-remembered configuration in a non-stimulated state (e.g., a low temperature state)), or two-way (having two remembered configurations, one upon being stimulated (e.g., a high temperature state), and the other non-stimulated (e.g., a low temperature state)) shape memory properties.

Shape memory polymers are a type of smart or intelligent material of particular relevance herein. SMPs may comprise thermoresponsive materials, meaning deformation can be induced and recovered through various temperature changes. Using a temperature-dependent process, some SMPs can be deformed into any shape, and when stimulated, regain a previous remembered or “memory” shape. Besides thermally activated SMPs, other SMPs exist which may be responsive to other external stimuli, such as electric or magnetic fields, light or radiation (e.g., laser light), or a change in pH. SMPs may have variable structures or compositions depending on their intended application.

SMPs are light, high in shape recovery ability, easy to manipulate, and economical as compared with SMAs. SMPs are generally characterized as phase segregated linear block co- polymers having a hard segment and a soft segment. The hard segment is typically crystalline, with a defined melting point, and the soft segment is typically amorphous, with a defined glass transition temperature. In some embodiments, however, the hard segment is amorphous and has a glass transition temperature rather than a melting point. In other embodiments, the soft segment is crystalline and has a melting point rather than a glass transition temperature. The melting point or glass transition temperature of the soft segment is substantially less than the melting point or glass transition temperature of the hard segment.

When a SMP is heated above its melting point or glass transition temperature of the hard segment, the material can be shaped. This (original or remembered) shape can be memorized by cooling the SMP below the melting point or glass transition temperature of the hard segment. When the shaped SMP is cooled below the melting point or glass transition temperature of the soft segment while the shape is deformed, a new (deformed or temporary) shape is fixed. The remembered shape is recovered by heating the material above the melting point or glass transition temperature of the soft segment but below the melting point or glass transition temperature of the hard segment. In another method for setting a deformed shape, the material is deformed at a temperature lower than the melting point or glass transition temperature of the soft segment, resulting in stress and strain being absorbed by the soft segment. When the material is heated above the melting point or glass transition temperature of the soft segment, but below the melting point (or glass transition temperature) of the hard segment, the stresses and strains are relieved and the material returns to its original shape. The recovery of the original shape, which is induced by an increase in temperature, is called the thermal shape memory effect. Properties that describe the shape memory capabilities of a material are the shape recovery of the original shape and the shape fixity of the temporary shape.

Generally speaking, the present invention describes a method and system for non-invasively and selectively controlling blood flow or blood flow rates through an arteriovenous access graft prosthesis at the site of implantation and anastomosis of the graft to provide an arteriovenous graft access, and for ultimately controlling or managing the blood supply through the target artery (the artery within the subject limb having the arterial end of the A/V graft prosthesis anastomosed thereto) to create a condition of improved vasculature within the subject limb. This condition of improved vasculature effectively reduces the potential for the onset of steal syndrome resulting from the formed arteriovenous graft access. By controlling the flow or flow rate of blood through the A/V access graft prosthesis, the overall blood flow through the A/V graft prosthesis may effectively be reduced, thus resulting in an effective increase in blood flow through the target artery as less blood is shunted through the A/V access graft prosthesis. Indeed, blood that would otherwise flow from the target artery into the A/V access graft prosthesis and then from the A/V graft prosthesis into the target vein (the vein within the subject limb having the venous end of the A/V graft prosthesis anastomosed thereto) as a result of the formed arteriovenous graft access (which, as discussed above, undesirably bypasses other important vessel portions of the subject limb, returning directly to the general circulation) is instead caused to continue through the artery finally reaching the remaining vessel portions of the subject limb prior. Stated differently, vessel portions of the subject limb supplied by the vasculature of the target artery and that are downstream from the A/V access graft prosthesis are caused to receive an increased blood supply at least somewhat alleviating, if not eliminating, many, if not all, of the symptoms associated with steal syndrome. As a result of the present invention, the vasculature that supplies the remaining vessel portions of the subject limb downstream from the site of the arteriovenous graft access, including the capillaries, may be dramatically enhanced or improved, and even optimized, all without sacrificing the integrity and/or diminishing the function of the arteriovenous graft access.

To achieve the foregoing, the present invention contemplates an arteriovenous access graft prosthesis for forming an arteriovenous graft access, having one or more constricting elements operable therewith, which constricting elements comprise a smart material that may be selectively activated in a non-invasive manner upon being exposed to one or more external stimuli. Upon activation, the constricting element constricts or shrinks to effectively reduce at least a portion of the inner luminal diameter of the tubular member of the A/V access graft prosthesis, thus permitting practitioners to selectively control blood flow through the A/V access graft prosthesis, and to ultimately enhance or optimize the vasculature supplying the remaining vessels of the subject limb. The present invention A/V access graft prosthesis, and the one or more constricting elements operable therewith, are discussed in greater detail below.

As will be apparent to those skilled in the art, the various exemplary present invention arteriovenous access graft prostheses provides several significant advantages over prior related arteriovenous access graft prostheses, some of which are recited here and throughout the following more detailed description. First, in some embodiments the present invention A/V access graft prosthesis provides multiple, selectable inner luminal diameters to specifically control the flow of blood through the lumen of the graft. Indeed, the constricting element may be configured to provide variable inner luminal diameters. For example, the degree of external stimulus may be specifically controlled and supplied, with the achieved inner luminal diameter being dependent upon the degree of external stimulus. Second, inner luminal diameter modifications to the tubular member may be made on-site, or rather after implantation or vessel anastomosis and under normal operating conditions, using non-invasive means and/or methods. Third, known invasive methods for treating steal syndrome and for alleviating or eliminating other symptoms caused by an insufficient supply of blood to the subject limb may be avoided. These known invasive methods include, but are not limited to, access ligation and/or banding of the graft or vessel distal to the graft to restrict blood flow through the graft. Fourth, although not required, the constricting element may be a low-aspect ratio component, meaning that it may be configured to comprise a short or diminutive longitudinal length as compared to the overall longitudinal length of the tubular member of the A/V access graft prosthesis. In this configuration, the constricting component may occupy a very small portion of surface area, or extend or span across only a short longitudinal distance, of the A/V access graft prosthesis. Fifth, different types and/or configurations of constricting elements may be used within the same A/V access graft prosthesis to achieve different results.

Each of the above-recited advantages will be apparent in light of the detailed description set forth below, with reference to the accompanying drawings. These advantages are not meant to be limiting in any way. Indeed, one skilled in the art will appreciate that other advantages may be realized, other than those specifically recited herein, upon practicing the present invention.

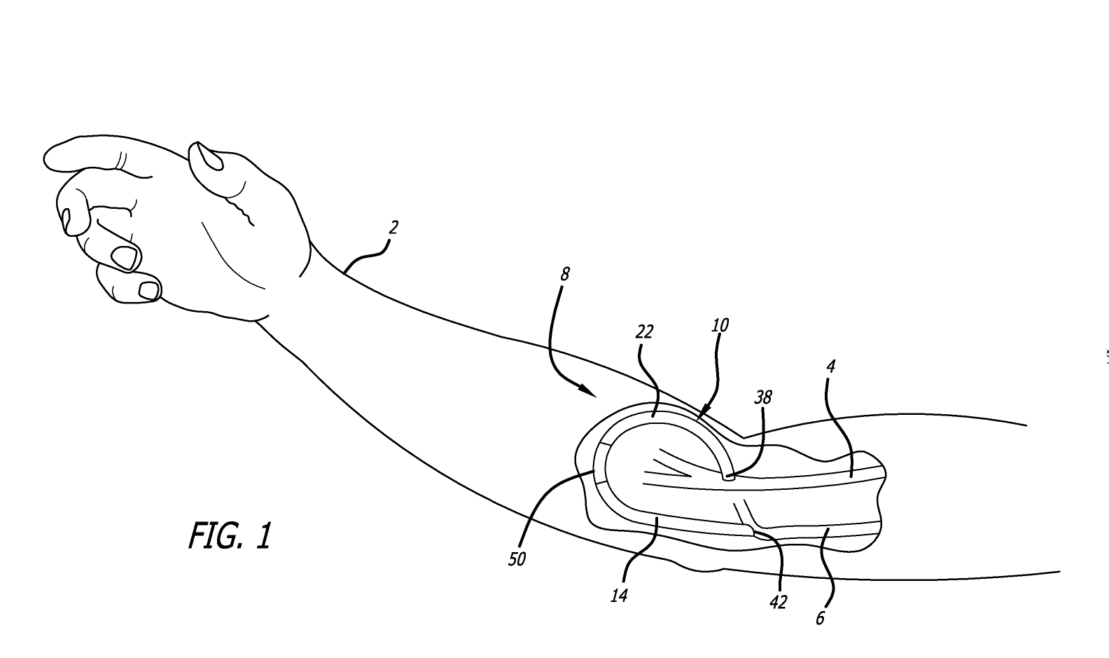

With reference to FIG. 1, illustrated is a graphical representation of a subject limb 2 in the form of a human arm, showing a portion of the vasculature having an artificial arteriovenous graft access 8 formed therein using an arteriovenous access graft prosthesis 10 configured in accordance with one exemplary embodiment of the present invention. The A/V access graft prosthesis 10 is shown as comprising a tubular member 14 terminating in an arterial end 38 adapted for arterial anastomosis, which arterial end 38 is shown as being anastomosed to a portion of a target artery 4 to split blood flow within the target artery between the A/V access graft prosthesis 10 and the target artery 4. Opposite the arterial end 38 is a venous end 42 adapted for venous anastomosis, which venous end 42 is shown as being anastomosed to a portion of a vein 6 to directly receive the portion of blood flowing through the A/V access graft prosthesis 10. In this configuration, the artificial arteriovenous graft access 8 is formed within the subject limb 2 for the purpose of providing a region or site of high blood flow access to the vasculature by a practitioner, and for facilitating hemodialysis treatment. One or both of the arterial end 38 and the venous end 42 may comprise a cuffed or flanged configuration as known in the art. The tubular member 14 and other components (e.g., any cuffed or flanged components) of the A/V access graft prosthesis 10 may be configured in a manner similar to other prior related vascular grafts, such as the Vectra® vascular access graft, the Venaflo® vascular graft, the Distaflo® vascular bypass graft, the Centerflex™ vascular graft and the IMPRA® Carboflo® series of vascular grafts, all of Bard Peripheral Vascular, Inc. (a division of C.R. Bard, Inc.).

Unlike prior related A/V access graft prostheses, the present invention A/V access graft prosthesis 10 further comprises means for constricting at least a portion of the luminal wall and reducing the inner luminal diameter of the tubular member 14 at the site of the A/V graft access 8, and with the A/V access graft prosthesis 10 functioning under normal operating conditions, even with means for constricting in a constricted state. The function of the means for constricting is to regulate blood flow through the target artery 4, and to control the volumetric blood flow rate being diverted into the target artery 4 as compared to the A/V access graft prosthesis 10. As shown, in one exemplary embodiment, means for constricting may comprise a constricting element 50 in the form of a band or annular ring independently operable with and disposed about the exterior surface 22 of the tubular member 14, which constricting element 50 is formed from a smart material and is configured to constrict a portion of the inner luminal diameter of the tubular member 14 upon being activated by an external stimulus (not shown). For example, the constricting element 50 may be formed of a smart material having a deformed, radially expanded diameter (preshrunk or deactivated) configuration and a remembered, radially reduced diameter (shrunk or activated) configuration, wherein the constricting element 50 may be selectively caused to shift or transition from the deformed, radially expanded diameter configuration or disposition to the remembered, radially reduced diameter or constricted configuration or disposition, as known in the art. It is noted herein that the constricting element 50 may comprise one-way or two-way smart material, with two-way smart material facilitating at least partial return to a remembered radially expanded configuration. Alternatively, the constricting element can incorporate two different smart materials to provide both selective reduction and expansion of the inner luminal diameter of the tubular member. Each material may comprise a different “memory” or remembered configuration, and could be configured to react differently to different external stimulus.

The means for constricting is operably related to the tubular member 14, meaning that it is secured about the luminal wall of the tubular member 14, such that the portion of the luminal wall in contact with the means for constricting is also caused to shrink or constrict, thereby causing the inner luminal diameter of the tubular member 14 to be reduced, or rather to cause at least a portion of the lumen to comprise a reduced diameter or cross-sectional area. The means for constricting may be located about the exterior surface, inner luminal surface and/or disposed between these two layers. In addition, the means for constricting may be located along a portion of the tubular member, or along the entire length of the tubular member (with the external stimulus being selectively applied to the means for constricting to activate and constrict all or a portion of the means for constricting).

FIG. 1 illustrates the constricting element in its deformed or unshrunk and inactivated condition permitting the maximum amount of blood flow through the A/V access graft prosthesis 10. In other words, the volumetric blood flow rate Q_{G} through the lumen of the tubular member 14 from the arterial end 38 to the venous end 42 is at a maximum. Conversely, the volumetric blood flow rate Q_{A} through the target artery 4 is at a minimum taking into account the presence of the A/V graft access. In other words, with the constricting element inactivated, a maximum amount of blood is diverted from the target artery 4 into the A/V access graft prosthesis 10. However, with means for constricting activated and in the constricted configuration, the volumetric blood flow rate Q_{G} through the A/V graft prosthesis 10 is reduced, with the volumetric blood flow rate Q_{A} through the target artery increasing. As blood passes into the lumen through the luminal opening in the arterial end 38 of the A/V access graft prosthesis 10 and reaches the reduced diameter portion or reduced cross-sectional area within the lumen, less blood is diverted from the target artery 4 into and through the A/V graft prosthesis 10, thus decreasing the overall volumetric blood flow rate Q_{G}. Naturally, therefore, as less blood is caused to be diverted into and through the A/V access graft prosthesis 10, the target artery 4 experiences an increase in blood flow, or volumetric blood flow rate Q_{A}.

The ratio of volumetric blood flow through the A/V access graft prosthesis 10 compared with the volumetric blood flow rate through the target artery 4, or Q_{G}:Q_{A}, will depend upon the degree to which the means for constricting reduces the diameter of the tubular member 14. In other words, the reduction in blood flow rate Q_{G} through the graft prosthesis will be proportional to the degree in which the inner luminal diameter is reduced as compared to its original diameter. By restricting blood flow and reducing the volumetric blood flow rate through the A/V graft prosthesis 10, the volumetric blood flow rate through the target artery 4 is conversely increased as more blood is channeled through the artery 4 rather than being diverted or shunted through the A/V graft access and the A/V graft prosthesis 10. An increase in volumetric blood flow through the target artery 4 allows a greater volume of blood to be supplied to the remaining vessels of the subject limb, thus preventing many of the complications and problems (e.g., Steal syndrome) caused by prior related A/V graft access formations.

Means for constricting may be located anywhere along the tubular member 14 between the arterial end 38 and the venous end 42. However, means for constricting will most likely be located in a position such that its function does not interfere with or jeopardize the integrity of either the arterial or venous anastomosis. As shown in FIG. 1, means for constricting is located approximately at a midpoint along the tubular member 14 between the arterial end 38 and venous end 42. However, locating the means for constricting at other locations along the tubular member other than the midpoint is also contemplated. In addition, means for constricting may be secured about the luminal wall of the tubular member using known methods, some of which are described in more detail below.

As indicated herein, the constricting element 50 (and others discussed herein) may comprise many different types of smart materials. Those skilled in the art will recognize the many different types of available smart materials that may be used to practice the present invention. Moreover, depending upon the type of smart material used to form the constricting element, different external stimuli may be used to bring about the transition of the constricting element to one or more remembered configurations (e.g., a remembered reduced diameter configuration, a remembered expanded diameter configuration, or both).

It is further contemplated herein that the degree of the applied stimulus may be selectively manipulated or controlled to specifically control the degree of transition or transformation of the constricting element. Indeed, it is contemplated that by controlling various aspects of the external stimulus, such as the intensity and duration of applied exposure, location of application, etc., that it is possible to control the degree of transformation of the constricting element (and also the location of constriction), thereby facilitating a range of available reduced diameter configurations within a single constricting element, and ultimately providing a variety of different volumetric flow rates through the A/V access graft prosthesis.

The present invention exemplary vascular graft prostheses, including means for constricting, may be formed from one or more materials having a suitable degree of biocompatibility. These biocompatible materials, or biomaterials, are generally described as materials, natural or man-made synthetic, that make up various biomedical devices intended to replace part of a living system or to function in intimate contact with living tissue. Biocompatible materials are intended to interface with biological systems to evaluate, treat, augment, perform or replace any tissue, organ or function of the body. Exemplary biocompatible materials for the tubular member include, but are in no way limited to, polytetrafluoroethylene (PTFE), expanded polytetrafluoroethylene (ePTFE), polyester, polyurethane, or fluoropolymers, such as perfluoroelastomers, and combinations thereof. While several suitable biocompatible materials exist in the art, the use of expanded polytetrafluoroethylene (ePTFE) as a nonviable, bio-inert barrier material is well known, and is a popular material selection for many graft prostheses. For example, the tubular member and any cuffed sections of the present invention vascular graft prosthesis may be formed from ePTFE, PTFE, or a combination of these. Depending upon the particular application, ePTFE may provide one or more advantages over other materials.

In addition, biocompatible smart materials may include, but are not limited to various types of SMAs, SMPs and others. Examples of suitable smart materials are known SMAs, as well as various SMPs, such as those disclosed in U.S. Publication No. 2004/0015187, which is incorporated by reference in its entirety herein.

It will be apparent to those skilled in the art that other biocompatible materials may exist that may be used, or that others being developed may also be used. As the focus of the present invention is not, per se, on the type of material used to form the one or more components of the vascular graft prosthesis, it is intended to be understood that other suitable biocompatible materials not mentioned herein are contemplated for use.

It is also contemplated that one or more bioactive agents may be incorporated into the components of the present invention vascular graft prosthesis. Exemplary bioactive agents include, but are not limited to, activated charcoal, carbon particles, graphite particles, vasodilator, anti-coagulants, such as, for example, warfarin and heparin. Other bio-active agents can also include, but are not limited to agents such as, for example, anti-proliferative/antimitotic agents including natural products such as vinca alkaloids (i.e. vinblastine, vincristine, and vinorelbine), paclitaxel, epidipodophyllotoxins (i.e. etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents such as G(GP) II.sub.b/III.sub.a inhibitors and vitronectin receptor antagonists; anti-proliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and analogs, streptozocin), trazenes-dacarbazinine (DTIC); anti-proliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine {cladribine}); platinum coordination complexes (cisplatin, carboplatin), procarbazine, hydroxyurea, mitotane, aminoglutethimide; hormones (i.e. estrogen); anti-coagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); anti-inflammatory: such as adrenocortical steroids (cortisol, cortisone, fludrocortisone, prednisone, prednisolone, 6.alpha.-methylprednisolone, triamcinolone, betamethasone, and dexamethasone), non-steroidal agents (salicylic acid derivatives i.e. aspirin; para-aminophenol derivatives i.e. acetominophen; indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives: (cyclosporine, tacrolimus (FK-506), sirolimus (rapamycin), azathioprine, mycophenolate mofetil); angiogenic agents: vascular endothelial growth factor (VEGF), fibroblast growth factor (FGF); angiotensin receptor blockers; nitric oxide donors; anti-sense oligionucleotides and combinations thereof; cell cycle inhibitors, mTOR inhibitors, and growth factor receptor signal transduction kinase inhibitors; retenoids; cyclin/CDK inhibitors; HMG co-enzyme reductase inhibitors (statins); and protease inhibitors.

In addition, it is noted that both the graft and the means for constricting may comprise a radiopaque or other material permitting these to be visible via ultrasound or other imaging methods. This may aid in efforts to appropriately expose the constricting element to an external stimulus.

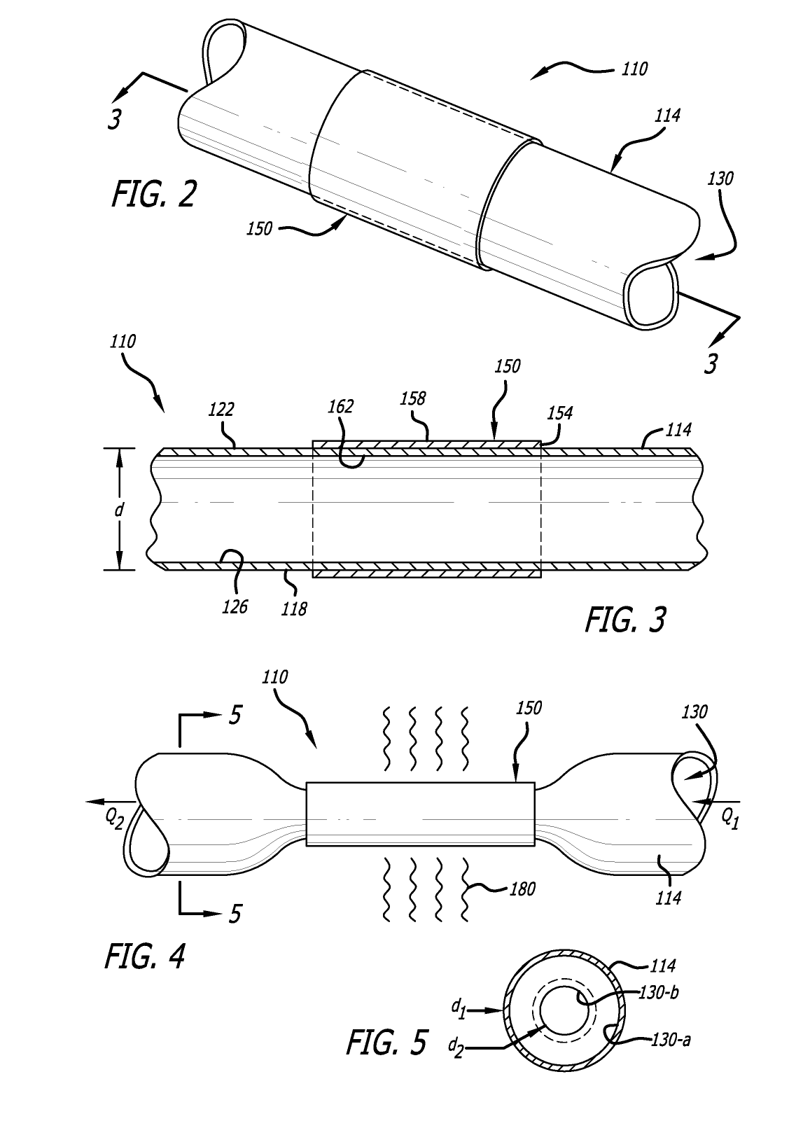

With reference to FIGS. 2-5, illustrated is an exemplary A/V access graft prosthesis 110 having a tubular body 114 defined by a luminal wall 118 having an exterior surface 122 and an inner luminal surface 126, which luminal wall 118 defines a lumen 130 for the passage of fluids, namely blood. Supported or disposed about the external surface 122 of the tubular member 114 is means for constricting the inner luminal diameter d of the tubular member 114 in the form of a constricting element 150 having a solid annular band or ring configuration that extends circumferentially around the tubular member 114. In this embodiment, the constricting element 150 comprises a tubular design having a wall structure 154 defining an exterior surface 158, an inner surface 162 and a lumen. The constricting element 150 comprises an inner diameter that is substantially the same as or slightly larger than the outside diameter of the tubular member 114 so as to permit the constricting element 150 to receive the tubular member 114, and to be secured about the exterior surface 122 of the tubular member 114 without adversely affecting the function of the A/V access graft prosthesis 110.

The constricting element 150 is formed of a smart material permitting the constricting element to transition from a radially extended configuration or disposition to a shrunk, radially constricted configuration upon being exposed to an external stimulus 180. The constricting element 150 may be designed to transition to a remembered configuration in response to an external stimulus 180 that is applied in a non-invasive manner, such as by applying thermal energy to the subject limb above the subcutaneous site of the implantation of the A/V access graft prosthesis 110 and formed A/V graft access. The type of smart material used to form the constricting element 150 may dictate the type of external stimulus that may be utilized to activate the constricting element 150. However, it is intended that the constricting element 150 be activated using non-invasive, externally applied measures.

The constricting element 150 may be secured to the tubular member 114 using any known securing means and/or method, such as via biocompatible adhesives, biocompatible mechanical fasteners or means (e.g., fasteners, staples, sutures, etc.), fusing or bonding, welding (e.g., using different techniques (heat, laser)) and any others recognized by those skilled in the art and their combinations. In the exemplary embodiment shown, the constricting element 150 is secured to the tubular member 114 using a biocompatible adhesive.

In the embodiment shown, the constricting element 150 may be exposed to an external stimulus 180 causing it to transition from a radially extended configuration to a reduced diameter configuration, thus ultimately causing the luminal wall 118 of the tubular member 114 to constrict, and thus altering the inner luminal diameter (see FIG. 4). With the constricting element 150 activated to reside in its constricted configuration, the lumen 130 comprises a non-uniform, inconsistent inner luminal diameter along the longitudinal length of the tubular member 114. As shown in FIG. 5, the overall luminal wall 118 defines a lumen 130-a having a greater or original inner luminal diameter d₁ defined by the luminal wall 118 residing in an original, unmodified configuration, and a lumen 130-b having a reduced or constricted diameter d₂ defined by the luminal wall 118 residing in a configuration having a reduced diameter or cross-sectional area as caused by the constricting element 150. As such, the overall volumetric flow rate Q_{G} through the lumen 130 of the A/V access graft prosthesis is restricted or reduced, with the volumetric flow rate Q_{A} within the artery increased.

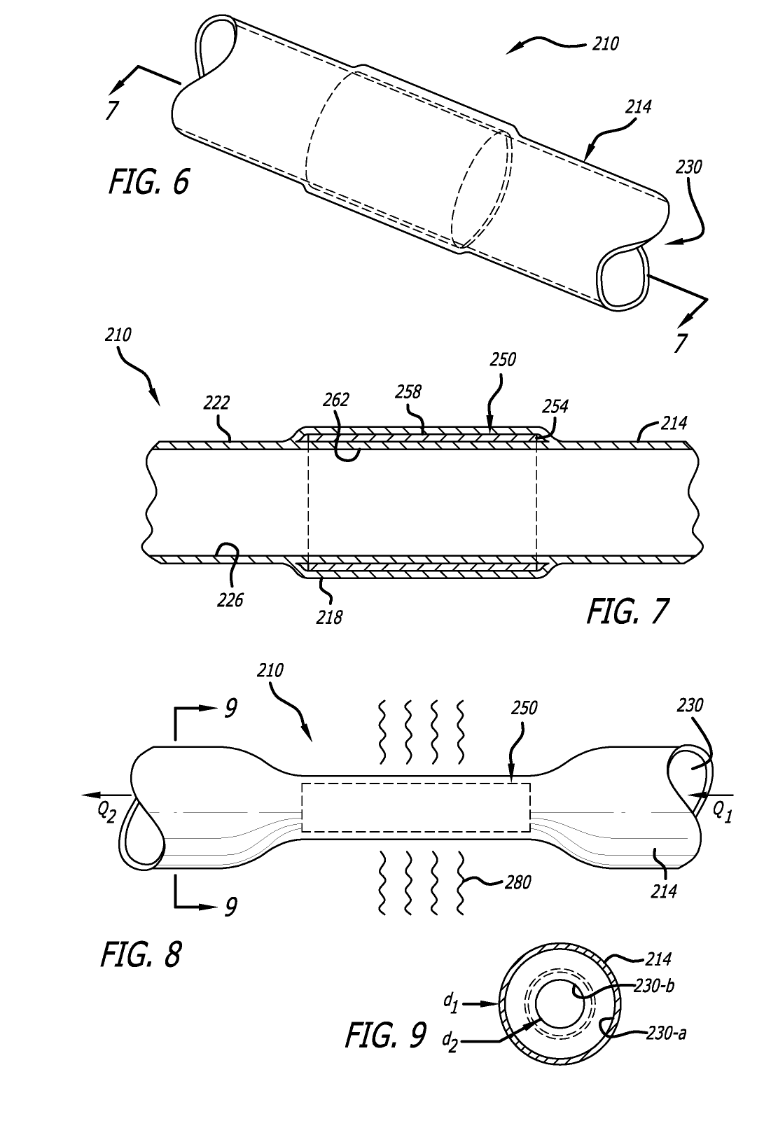

Referring now to FIGS. 6-9, illustrated is another exemplary A/V access graft prosthesis 210 having a tubular body 214 defined by a luminal wall 218 having an exterior surface 222 and an inner luminal surface 226, which luminal wall 218 defines a lumen 230. The A/V access graft prosthesis 210 is similar in many respects to the A/V access graft prosthesis 110 discussed above and shown in FIGS. 2-5. However, unlike this previous embodiment, the A/V access graft prosthesis 210 comprises means for constricting the inner luminal diameter of the tubular member 214 in the form of a constricting element 250 configured as band or strip of smart material enclosed or encased within (e.g., sandwiched between) the luminal wall 218 of the tubular member 214 between the external surface 222 and the inner luminal surface 226, such that no part of the constricting element 250 is exposed.

The constricting element 250 comprises a tubular design having a wall structure 254 defining an exterior surface 258, an inner surface 262 and a lumen. The constricting element 250 comprises inner and outer diameters configured to permit its disposal between the external and inner luminal surfaces of the tubular member 214. The luminal wall 218 functions to secure the constricting element 250 in place. However, additional biocompatible securing means, such as biocompatible adhesives and/or mechanical fasteners, may be used to ensure that the constricting element 250 remains in place.

The constricting element 250 is shown as being formed of a smart material permitting the constricting element 250 to transition from a radially extended configuration or disposition to a shrunk, radially constricted configuration upon being exposed to an external stimulus 280 (see FIG. 8). The function of the external stimulus and the resultant response of the constricting element 250 exposed thereto is as described above. As described above, with the constricting element 250 activated to reside in its constricted configuration, the lumen 230 comprises a non-uniform, inconsistent inner luminal diameter along the longitudinal length of the tubular member 214. As shown in FIG. 9, the overall luminal wall 218 defines a lumen 230-a having a greater or original inner luminal diameter d₁ defined by the luminal wall 218 residing in an original, unmodified configuration, and a lumen 230-b having a reduced or constricted diameter d₂ defined by the luminal wall 218 residing in a configuration having a reduced diameter or cross-sectional area as caused by the constricting element 250. As such, the overall volumetric flow rate Q_{G} through the lumen 230 of the A/V access graft prosthesis is restricted or reduced, with the volumetric flow rate Q_{A} through the target artery increased.

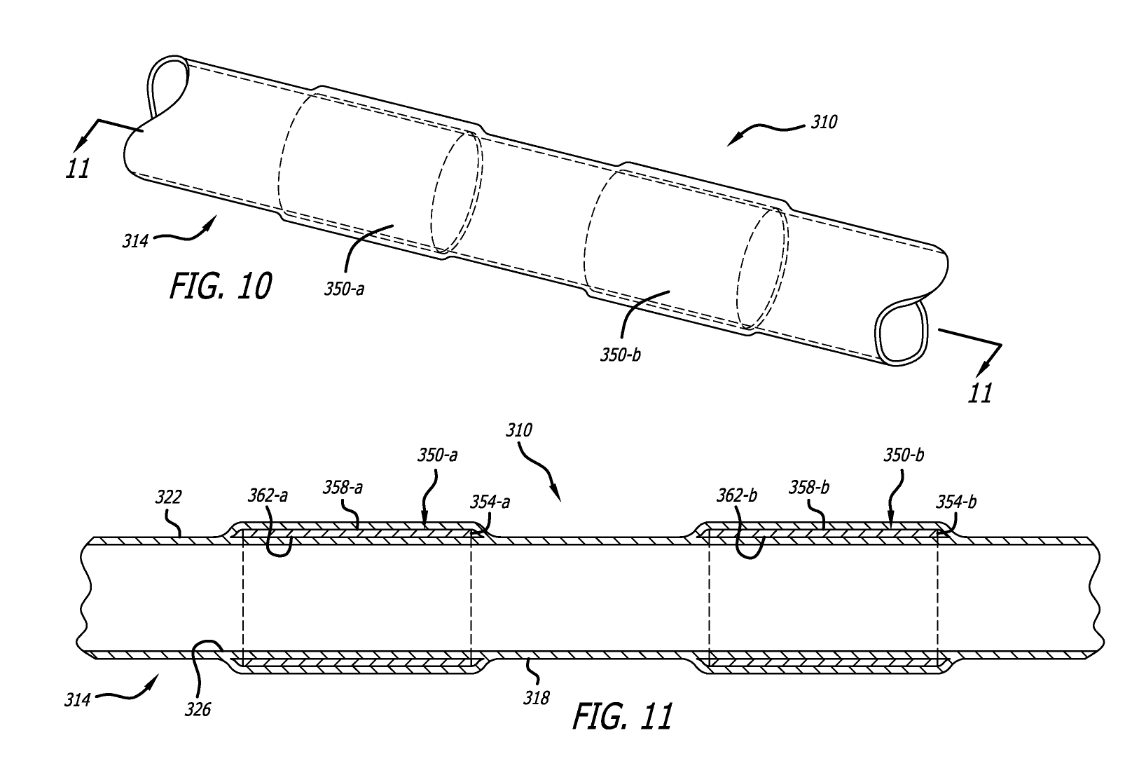

With reference to FIGS. 10-13, illustrated is still another exemplary A/V access graft prosthesis 310 having a tubular body 314 defined by a luminal wall 318 having an exterior surface 322 and an inner luminal surface 326, which luminal wall 318 defines a lumen 330. This embodiment is similar in many respects to the A/V access graft 210 described above and shown in FIGS. 6-9, with one notable difference. Rather than employing a single constricting element, the A/V access graft prosthesis 310 comprises a plurality of constricting elements, shown as first constricting element 350-a and second constricting element 350-b, each one capable of providing a different reduced diameter or cross-sectional area within the lumen 330 of the tubular member 314 in a radially constricted configuration. The several constricting elements may be specifically tuned by varying the element ratios within the material makeup. The plurality of constricting elements 350-a and 350-b are each shown as comprising a band or strip of smart material encased or enclosed within the luminal wall 318 of the tubular member 314 between the external surface 322 and the inner luminal surface 326, such that no part of the constricting elements are exposed. The constricting elements 350-a and 350-b each comprise a wall structure defining an exterior surface and an interior surface (see walls 354-a and 354-b, exterior surfaces 358-a and 358-b, and interior surfaces 362-a and 362-b, respectively). In addition, the first constricting element 350-a may be placed immediately adjacent to, or in a spaced apart position with respect to, the second constricting element 350-b. One consideration may be that, depending upon the type of constricting elements used, the degree of potential reduction of each or a combination of these and/or other factors, it may be desirable to appropriately space the plurality of constricting elements apart from one another along the longitudinal axis of the tubular member 314 a sufficient distance so as to not interfere with or jeopardize the integrity of the tubular member 314. Indeed, it may be advantageous to provide a sufficient distance between constricting elements to accommodate a suitable degree of distortion or deformation within the tubular member 314, which may occur upon activation of either or both of the constricting elements.

The plurality of constricting elements function to provide the A/V access graft prosthesis 310 with a plurality of different selectable “zones” or states of reduced inner luminal diameter, or reduced luminal cross-sectional area, each of which may generate different desired volumetric flow rates through the lumen 330. In other words, it is intended that each different constricting element provide a different reduced inner luminal diameter when activated and caused to transition to the constricted radial configuration. Providing multiple constricting elements gives practitioners several different options for reducing volumetric blood flow at the A/V graft access site within a single A/V access graft prosthesis, as well as increasing volumetric blood flow through the target artery. Indeed, depending upon the severity of manifested symptoms for a particular patient, the practitioner can selectively activate a particular constricting element, or particular combination of constricting elements, most appropriate for treatment. In addition, a single A/V graft prosthesis design may be configured to be more universally acceptable, and to accommodate a larger percentage of patients, thus potentially reducing manufacturing costs, patient care center costs, and patient expenses.

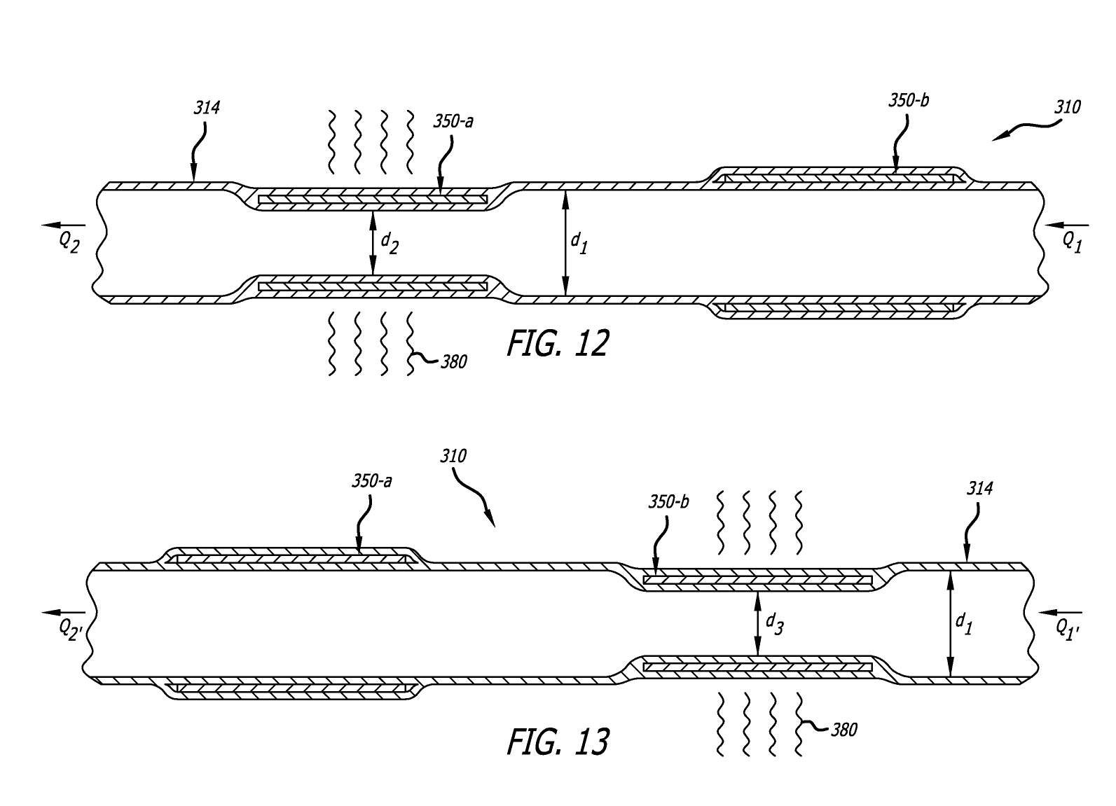

To illustrate the concepts discussed above, as shown in FIGS. 12 and 13, the A/V access graft prosthesis 310 may be configured to comprise two constricting elements, namely constricting elements 350-a and 350-b, that operate to provide the A/V access graft prosthesis 310 with three available and selectable reduced diameter states or configurations for treating different degrees of severity of steal syndrome and/or different degrees of other possible manifested symptoms within the subject limb. The first selectable state may comprise activation of the first constricting element 350-a (by exposing the constricting element 350-a to an external stimulus 380), which may represent the least available reduction in inner luminal diameter (see diameter d₂ as compared with the diameter d₁ of the tubular member 314) and the minimum reduction of volumetric flow rate through the lumen 330 of the tubular member. The first state may be selected specifically for treatment of mild cases of steal syndrome and/or relatively mild or faint manifested symptoms in the subject limb. The second selectable state may comprise activation of the second constricting element 350-b (by exposing the constricting element 350-b to an external stimulus 380), which may represent the greatest available reduction in inner luminal diameter (see diameter d₃ as compared with the diameter d₁ of the tubular member 314, and the diameter d₂ created by the constricting element 350-a (diameter d₃ being smaller than diameter d₂)) and a moderate reduction of volumetric flow rate through the lumen 330. The second state may be selected for treatment of intermediate cases of steal syndrome and/or moderate manifested symptoms in the subject limb. The third selectable state may comprise the concurrent activation of both the first and second constricting elements 350-a and 350-b (by exposing each of the constricting elements 350-a and 350-b to an external stimulus 380), which may represent the maximum available overall reduction of volumetric flow rate through the lumen 330 of the tubular member 314 for treatment of severe cases of steal syndrome and/or acute symptoms in the subject limb. Although the inner luminal diameter is no smaller in this third selectable state than it is in the second selectable state, the concurrent activation of both constricting elements 350-a and 350-b, permits these to act in concert with one another to effectively provide a cumulative reduction in volumetric flow rate through the lumen 330.

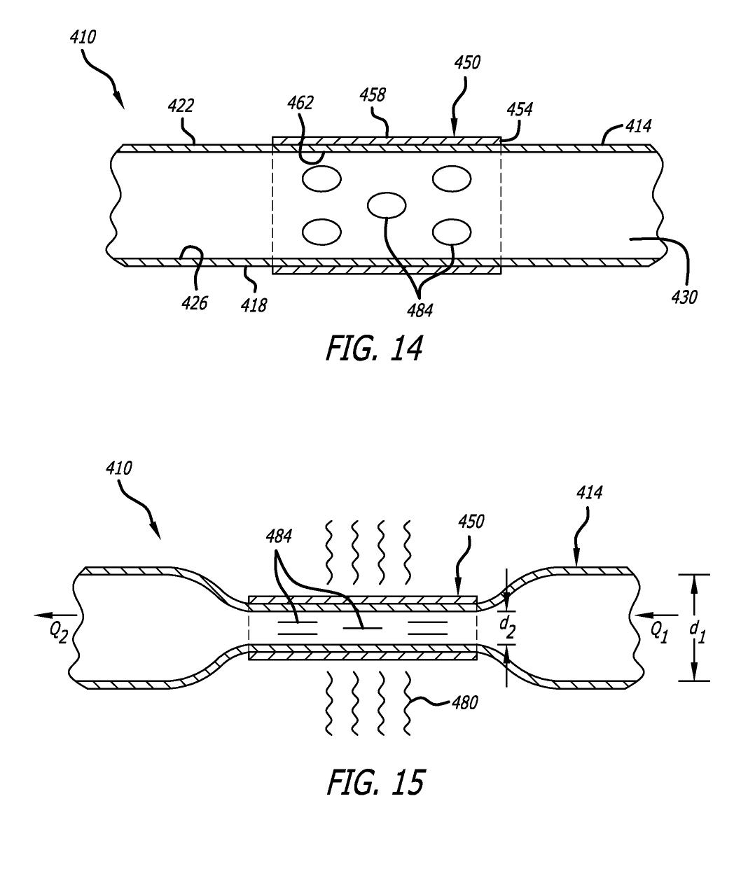

With reference to FIGS. 14 and 15, illustrated is still another exemplary embodiment of an A/V access graft prosthesis 410 comprising a tubular member 414 having a luminal wall 418 defining an exterior surface 422, an inner luminal surface 426 and a lumen 430. The A/V access graft prosthesis 410 is similar to the A/V access graft prosthesis 110 described above and shown in FIGS. 2-5. Indeed, the A/V access graft prosthesis 410 comprises means for constricting in the form of a constricting element 450 having an annular band or ring-like configuration configured to be disposed about the exterior surface 422 of the tubular member, and made of a smart material. However, the A/V access graft prosthesis 410 comprises at least one difference. Formed in the luminal wall 418 are a plurality of apertures 484, each of which are designed and configured to at least partially collapse or close upon activation of the constricting element 450 to provide a reduced inner luminal diameter or cross-sectional area to provide a reduced volumetric flow rate as compared to an original volumetric flow rate. The function of the apertures 484 is to provide an element of forgiveness within the luminal wall 418, to reduce the potential for surface irregularities or anomalies along the inner luminal surface 426, particularly about the region of reduced inner luminal diameter, that may be induced upon transitioning the constricting element 450 to provide a reduced inner luminal diameter.

Surface irregularities may manifest themselves in the form of wrinkles, folds, peaks, valleys, etc. within the luminal wall 418. Indeed, depending upon the transition differential existing between the radially expanded configuration and the radially reduced configuration, the luminal wall 418 may deform or distort in an undesirable manner, causing or one or more irregularities to be induced along the inner luminal surface 426. Such surface irregularities may significantly affect the performance of the A/V access graft prosthesis. For example, formation of surface irregularities along the inner luminal surface 426 could adversely affect the hemodynamics of the A/V access graft prosthesis, which could increase the potential for the onset of intimal hyperplasia and/or thrombosis, each of which would operate to decrease the patency of the A/V graft access.

Each of the apertures is strategically designed to allow the luminal wall 418 to, in essence, collapse upon itself and to eliminate the potential for buckling, wrinkling, folding, corrugate, etc., anything that would be considered an undesirable surface anomaly. The apertures 484 may comprise any size and geometric configuration. In addition, any number of apertures may be formed in the luminal wall 418, as needed or desired. The apertures may be present in the luminal wall 418 as the inner surface 462 of the constricting element 450 is intended to seal the apertures 484. Indeed, the constricting element 450 is intended to be secured or disposed about the luminal wall 418 (either about the exterior surface 422 of the tubular member 414, between layers of the luminal wall 418, or about the inner luminal surface 426 of the tubular member 414), and to comprise, at least partially (in the region about the apertures) a solid surface configuration. In the event one or more apertures 484 are desired, the constricting element 450 may be secured in a manner so as to ensure sealing of the apertures by the wall 454 of the constricting element 450, thus preserving the integrity of the tubular member, and eliminating the potential for leakage through the apertures 484.

In the embodiment shown, the apertures 484 are in the form of a plurality of ellipses, each of which are present in a pattern within the luminal wall 418. Upon exposing the constricting element 450 to an external stimulus 480 to effect transition of the constricting element 450 to a reduced diameter configuration (see FIG. 15, and diameter d₂ as compared to diameter d₁), each of the apertures 484 are caused to collapse upon themselves to accommodate any deformation or distortion within the luminal wall 418. Although the apertures 484 are shown as being completely collapsed, this should not be limiting in any way. Partial collapse by the apertures is also contemplated, depending upon the degree of radial reduction within the constricting element 450, the configuration of the apertures, and/or other factors that will be obvious to those skilled in the art. Moreover, as will also be obvious to those skilled in the art, the apertures are not required to be arranged in a pattern, to each comprise the same size or geometry, or to exist in any given number.

It is noted herein, although not shown in the drawings, that portions of the tubular member intended to receive and support a constricting element may be pre-stretched, with the pre-stretched state being intended to accommodate the constricting element in its deformed, radially expanded diameter state. Upon transitioning of the constricting element to the remembered, radially reduced diameter configuration, the tubular member would resist wrinkling, buckling, folding, etc. as the material making up the tubular member would be permitted to return to a relaxed, non-shrunk state.

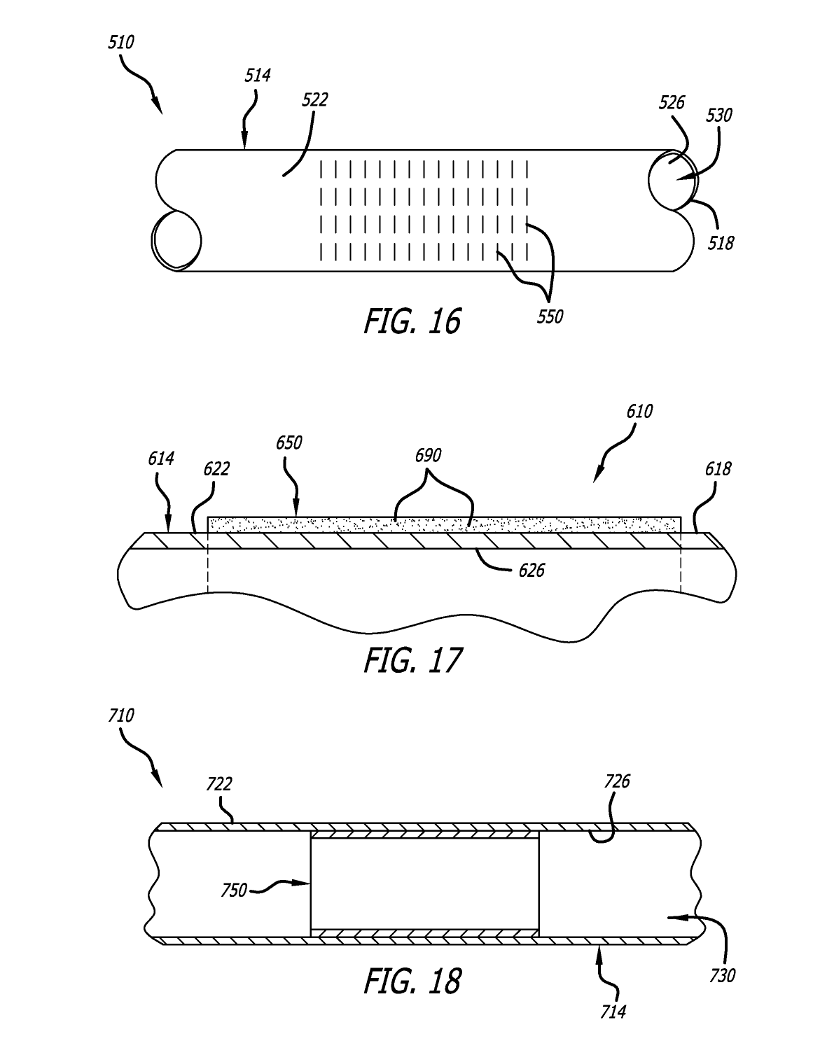

FIG. 16 illustrates an A/V access graft prosthesis in accordance with another exemplary embodiment of the present invention. In this particular embodiment, the A/V access graft prosthesis 510 comprises a tubular member 514 having a luminal wall 518 defining an exterior surface 522, an inner luminal surface 526 and a lumen 530. The A/V access graft prosthesis 510 further comprises means for constricting the inner luminal diameter of the tubular member 514 in the form of a plurality of wire-like constricting elements 550 oriented transversely about the luminal wall 518 of the tubular member 514. In one aspect, the wire-like constricting elements 550 may be impregnated into the exterior surface 522 of the tubular member. In another aspect, the wire-like constricting elements 550 may be encased between layers of the luminal wall 518.

Upon being exposed to an external stimulus (not shown), each of the wire-like constricting elements 550 transition from an extended length to a reduced length, thus causing the luminal wall to constrict to generate a reduced inner luminal diameter or cross-sectional area. As such, each of the wire-like constricting elements 550 are formed of a smart material, and are configured to comprise a deformed extended configuration, and a shorter “remembered” configuration. Although comprising a different configuration, the wire-like constricting elements 550 effectively function in a similar manner as the several constricting elements discussed above, namely to reduce the inner luminal diameter of at least a portion of the tubular member 514 for the purpose of reduce the volumetric flow rate Q_{G} through the A/V graft prosthesis 510. However, the plurality of wire-like constricting elements 550 are intended to work in concert with one another to effectuate a reduction in the inner luminal diameter of the tubular member 514.

The wire-like constricting elements 550 may comprise a linear configuration or a curved configuration matching the curvature of the luminal wall 518 of the tubular member. The wire-like constricting elements 550 may also be present in different quantities than shown, as will be obvious to one skilled in the art.

FIG. 17 illustrates an exemplary A/V access graft prosthesis 610 that operates similar to the several A/V access graft prostheses discussed above to reduce at least a portion of the inner luminal diameter of a tubular member 614. The A/V access graft prosthesis 610 comprises a tubular member 614 having a luminal wall 618 defining an exterior surface 622 and an inner luminal surface 626. Disposed about the exterior surface 622 is means for constricting at least a portion of the luminal wall and reducing the inner luminal diameter of the tubular member 614, which is similar in many respects to the various other embodiments discussed above.

In this particular embodiment however, the means for constricting comprises a constricting element 650 formed of a thermoresponsive smart material having a plurality of carbon nanotubes (tiny fibers of pure carbon), embedded therein. Incorporating carbon nanotubes within the thermoresponsive smart material provides the distinct advantage of being able to utilize an external stimulus other than external thermal energy or heat to activate the smart material and cause the constricting element 650 to transition from its original radially expanded configuration to its “remembered” reduced diameter configuration. This is not to say that the thermoresponsive smart material does not require thermal energy to effectuate its transition. Indeed it does. However, the presence of the several carbon nanotubes within the smart material permits the external stimulus to be something other than an externally generated thermal energy stimulus. Carbon nanotubes, when exposed to light, such as laser light, near-infrared light, etc., generate excess energy in the form of thermal energy or heat. As such, when the constricting element 650, having the carbon nanotubes embedded therein, is exposed to light capable of causing the carbon nanotubes to generate thermal energy, this thermal energy is effectively transferred, via thermal conduction, to the surrounding smart material composition, thus providing the necessary thermal energy needed to initiate the transition of the constricting element 650 to its reduced diameter configuration. This particular method is advantageous for many reasons, one of which is that tissue surrounding the A/V graft access is not exposed to high levels of thermal energy from an external stimulus. Rather, the external stimulus may simply comprise a light source that is completely harmless to the tissue.

The level of thermal energy generated by the carbon nanotubes, and therefore the degree of transformation of the constricting element, may be specifically controlled by varying the intensity and duration of light incident on the constricting element 650. In addition, the size, number or density of carbon nanotubes within the smart material may be specifically controlled to control the degree of transformation of the constricting element. Those skilled in the art will recognize the several different material compositions that can be made available to produce different and specific transformation effects.

It is noted that any of the exemplary constricting elements discussed herein may comprise carbon nanotubes in their makeup. In addition, it is further noted herein that the present invention contemplates the use of carbon nanotubes in other biomedical implantable devices, such as stents and stent-grafts.

FIG. 18 illustrates another exemplary embodiment of the present invention, wherein an A/V access graft prosthesis 710 comprises a tubular member 714 having a luminal wall 718 defining an exterior surface 722, an inner luminal surface 726 and a lumen 730. Disposed about the inner luminal surface 726 is means for constricting at least a portion of the luminal wall and reducing the inner luminal diameter of the tubular member 714, which is similar in many respects to the various other embodiments discussed above. The means for constricting is in the form of a constricting element 750 having a band or ring-like configuration with an outside diameter substantially similar as the diameter of the inner luminal surface 726 so as to be fittable within the lumen 730. The constricting element comprises a smart material that is secured to the inner luminal surface 726 using any known means as discussed herein. Upon activation of the constricting element 750 to cause it to transition from its original, deformed radially expanded configuration to its remembered radially reduced diameter configuration, the constricting element 750 essentially pulls or draws the luminal wall 718 inward, thus reducing the diameter of the lumen 730 of the tubular member 714.

It is noted that the present invention concept of constricting the inner luminal diameter of a vascular graft, as discussed herein, may be applicable to other types of vascular graft prostheses other than A/V access graft prostheses. For example, different types of peripheral bypass or other vascular grafts may benefit from an element operable to constrict their inner luminal diameter for one or more purposes to be recognized by those skilled in the art. Although the discussion herein centers around different exemplary A/V access graft prostheses, this particular vascular graft type is not intended to be limiting in any way.

The foregoing detailed description describes the invention with reference to specific exemplary embodiments. However, it will be appreciated that various modifications and changes can be made without departing from the scope of the present invention as set forth in the appended claims. The detailed description and accompanying drawings are to be regarded as merely illustrative, rather than as restrictive, and all such modifications or changes, if any, are intended to fall within the scope of the present invention as described and set forth herein.

More specifically, while illustrative exemplary embodiments of the invention have been described herein, the present invention is not limited to these embodiments, but includes any and all embodiments having modifications, omissions, combinations (e.g., of aspects across various embodiments), adaptations and/or alterations as would be appreciated by those skilled in the art based on the foregoing detailed description. The limitations in the claims are to be interpreted broadly based on the language employed in the claims and not limited to examples described in the foregoing detailed description or during the prosecution of the application, which examples are to be construed as non-exclusive. For example, in the present disclosure, the term “preferably” is non-exclusive where it is intended to mean “preferably, but not limited to.” Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given above.

What is claimed is:

1. A vascular graft prosthesis anastomosed to a target vessel within a subject limb, the vascular graft prosthesis comprising:
a tubular member having a luminal wall defining a lumen and an inner luminal diameter; and
a constricting element in contact with the luminal wall, the constricting element comprising a smart material operable by an external stimulus, the constricting element transitioning from a first diameter to a second diameter smaller than the first diameter upon application of said external stimulus, the transitioning of the constricting element to the second diameter decreasing the inner luminal diameter.

2. The vascular graft prosthesis according to claim 1, wherein the constricting element comprises an annular band disposed about a surface of the tubular member.

3. The vascular graft prosthesis according to claim 1, wherein the constricting element is embedded in the luminal wall of the tubular member.

4. The vascular graft prosthesis according to claim 1, wherein the constricting element comprises a first constricting element spaced apart from a second constricting element along a longitudinal axis of the tubular member.

5. The vascular graft prosthesis according to claim 4, wherein application of the external stimulus transitions the first constricting element from the first diameter to the second diameter, and transitions the second constricting element from the first diameter to a third diameter smaller than the second diameter.

6. The vascular graft prosthesis according to claim 4, wherein the first and second constricting elements are embedded in the luminal wall of the tubular member.

7. The vascular graft prosthesis according to claim 4, wherein the constricting element further comprises a third constricting element spaced apart from the first and second constricting elements along a longitudinal axis of the tubular member.

8. The vascular graft prosthesis according to claim 7, wherein application of the external stimulus transitions the first constricting element from the first diameter to the second diameter, transitions the second constricting element from the first diameter to a third diameter different from the second diameter, and transitions the third constricting element from the first diameter to a fourth diameter different from the second diameter and the third diameter.

9. The vascular graft prosthesis according to claim 1, wherein the luminal wall comprises a plurality of apertures, the constricting element disposed in a lumen sealing position around the apertures.

10. The vascular graft prosthesis according to claim 9, wherein transitioning of the constricting element to the second diameter substantially closes the apertures.

11. The vascular graft prosthesis according to claim 1, wherein the constricting element includes a plurality of wire-like elements oriented transversely with respect to a longitudinal axis of the tubular member.

12. The vascular graft prosthesis according to claim 1, wherein the smart material is a thermoresponsive smart material, the constricting element further comprising a plurality of carbon nanotubes.

13. The vascular graft prosthesis according to claim 1, wherein the smart material has a deformed, expanded configuration at the first diameter, and a remembered, reduced configuration at the second diameter.

14. The vascular graft prosthesis according to claim 1, wherein the smart material is selected from the group consisting essentially of shape memory alloys, shape memory polymers, piezoelectric materials, magnetic shape memory alloys, and combinations thereof.

15. The vascular graft prosthesis according to claim 1, wherein the constricting element is positioned along a longitudinal axis of the tubular member at approximately a midpoint between a proximal end of the tubular member and a distal end of the tubular member.

16. A vascular graft prosthesis, comprising:
a tubular member having a luminal wall defining a lumen and an inner luminal diameter, the tubular member including an arterial end for attachment to an artery and a venous end for attachment to a vein; and
a constricting element in contact with the luminal wall, the constricting element comprising a shape memory polymer transitioning from a first deformed configuration to a second remembered configuration upon application of an external stimulus, the constricting element decreasing the inner luminal diameter as the shape memory polymer transitions to the second remembered configuration.

17. The vascular graft prosthesis according to claim 16, further comprising a bioactive agent incorporated into the luminal wall.

18. The vascular graft prosthesis according to claim 16, wherein the constricting element is encapsulated by a biocompatible material.

19. (canceled)

20. A method of regulating blood flow in a patient through the use of an arteriovenous access graft prosthesis, comprising:
providing an arteriovenous access graft prosthesis comprising a tubular member having a luminal wall defining a lumen and a constricting element in contact with the luminal wall, the constricting element comprising a smart material transitioning from a first deformed configuration to a second remembered configuration upon application of an external stimulus, the constricting element decreasing a diameter of the lumen during transition to the second remembered configuration;
implanting the graft prosthesis in a patient by attaching an arterial end of the graft prosthesis to an artery of the patient and a venous end of the graft prosthesis to a vein of the patient;
applying said external stimulus to the approximate location of the constricting element within the patient's body to decrease the lumen diameter; and
removing said external stimulus to increase the lumen diameter.
